(19) United States
(12) Patent Application Publication
(10) Pub. No.: US 2021/0283174 A1
(43) Pub. Date: Sep. 16, 2021

(54) OXYGENATED EMULSION FOR TREATMENT OF OCULAR INJURY

(71) Applicants: The Schepens Eye Research Institute, Inc., Boston, MA (US); Coruna Medical LLC, Longmont, CO (US)

(72) Inventors: Jia Yin, Winchester, MA (US); Kate Pate, Golden, CO (US); Sharon Lake, Longmont, CO (US); Mark Fontenot, Lafayette, LA (US)

(21) Appl. No.: 17/200,253

(22) Filed: Mar. 12, 2021

Related U.S. Application Data
(63) Continuation-in-part of application No. 17/172,403, filed on Feb. 10, 2021.
(60) Provisional application No. 63/133,865, filed on Jan. 5, 2021.
(60) Provisional application No. 62/989,354, filed on Mar. 13, 2020.

(51) Int. Cl. A61K 33/00; A61K 9/107; A61K 47/06; A61K 47/44; A61K 47/24; A61K 9/00; A61K 9/12; A61P 27/02

(52) U.S. Cl. CPC A61K 33/00; A61K 9/107; A61K 47/06; A61P 27/02; A61K 47/24; A61K 9/0048; A61K 9/12; A61K 47/44

(57) ABSTRACT

An oxygenated material for application to an alkali burn in an ocular region. The oxygenated material includes a supersaturated oxygen emulsion (SSOE) including perfluorodecalin (PFD) homogenized with a phospholipid and an emulsifying wax. The SSOE comprises a viscosity configured to deliver the SSOE to a site of the alkali burn and sustain a partial pressure of oxygen at the site at a level a plurality of times greater than an ambient partial pressure of oxygen for a plurality of hours. A pressurized dispensing canister is configured to house and dispense the SSOE directly on the site.

### RELATED APPLICATIONS

This application claims the benefit of United States (US) Patent Application No. 62/989,354, filed Mar. 13, 2020.

This application claims the benefit of U.S. Patent Application No. 63/133,865, filed Jan. 5, 2021.

This application is a continuation in part of U.S. patent application Ser. No. 17/172,403, filed Feb. 10, 2021.

### GOVERNMENT LICENSE RIGHTS

This invention was made with government support under W81XWH-17-C-0008 awarded by the US Army Medical Research and Materiel Command. The government has certain rights in the invention.

### TECHNICAL FIELD

The present invention relates generally to systems and methods for treatment of ocular injury.

### BACKGROUND

Injuries to the eye include abrasion, thermal burns, and chemical burns (e.g., acidic burns, alkali burns, etc.), and cause tissue hypoxia, inflammation, and vision loss with limited treatment options. Chemical burns for example, which represent up to 20% of eye injuries, occur in household, industrial, and military settings and are considered true ocular emergency that requires immediate evaluation and treatment. The impact of severe chemical burns on the eye is profound, including eyelid skin deformity, corneal opacification and neovascularization, anterior chamber inflammation and exudation (hypopyon), cataract formation, and even damage to the posterior segment including the retina and optic nerve. Conventional treatments focus on rapid removal of chemicals via irrigation and reducing subsequent inflammation. Detrimental damage to the eye occurs despite these conventional treatments, and often results in permanent vision loss and blindness. The medical community therefore lacks any targeted therapy for these eye injuries resulting in a significant unmet medical need. Thus, there is a critical need to address ocular injuries through measures that promote tissue preservation and wound healing.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE FIGURES

FIG. 1 is a flow diagram of a method of forming the supersaturated oxygen emulsion (SSOE), under an embodiment.

FIG. 2 is a flow diagram of a method of forming and packaging the SSOE, under an embodiment.

FIG. 3 shows a method of treating an ocular injury (e.g., alkali burn) by applying the SSOE, under an embodiment.

### DETAILED DESCRIPTION

Oxygen plays a vital role in wound healing, and oxygen therapy is shown herein to be beneficial in treating acute ocular injuries. Supersaturated Oxygen Emulsion (SSOE) is a biocompatible emulsion comprising perfluorodecalin (PFD), and is manufactured in hyperbaric conditions and configured to rapidly and continuously release oxygen. SSOE technology harnesses high levels of oxygen with proven stability over time and delivers oxygen to targeted tissue with rapid and sustainable release. Embodiments described herein include the development of an ophthalmic supersaturated oxygen emulsion (SSOE) comprising PFD (referred to herein as “SSOE”), and present results of methods evaluating its safety and efficacy in mitigating acute ocular injury in ocular regions including one or more of corneal tissue, limbal tissue, ocular adnexal tissue, retinal tissue, ocular nerve tissue, and vitreous compartments.

The ophthalmic SSOE of embodiments described herein is demonstrated to be safe and to reverse tissue hypoxia in the eye, for example after injury in mice following which a single application of SSOE was efficacious in reducing ocular inflammation, opacity and cataract formation. More specifically, oxygen therapy as described herein is demonstrated to improve limbal ischemia, accelerated epithelialization, increased corneal transparency, and decreased corneal vascularization as compared to conventional medical treatment. Consequently, increasing the partial pressure of oxygen at the corneal surface with a single topical application of SSOE as an ophthalmic emulsion at time of injury is efficacious in mitigating the tissue damage and may lead to the prevention of neovascularization and corneal clouding, improved tissue preservation, and enhanced wound healing following injury.

Perfluorocarbons (PFCs) are inert chemicals with great biocompatibility and oxygen-binding capacity. At room temperature, the solubility of oxygen (02) is 40% or more in PFC, compared to 2.5% in water, 2.5% in plasma, and 20% in whole blood. Of all PFCs, PFD has been most widely used in medical applications due to its high oxygen-carrying capacity, dissolving 49 mL of oxygen per 100 mL of PFD at room temperature and pressure. PFCs including PFD are hydrophobic and therefore must be emulsified to promote water solubility. Embodiments described herein include use of two surfactants, Phospholipon 90H and Polawax (e.g., fixed proportion 5.0:1.8), to emulsify the PFD emulsion but are not so limited. Perfluorodecalin was distributed by Mel-Co® (Coachella Valley, Calif.). Phospholipon 90H was from American Lecithin Company (Oxford, Conn.). Polawax was from Croda Inc (Edison, N.J.).

Embodiments described herein harvested the beneficial effect of oxygen in treatment of ocular tissue injuries through topical delivery of oxygen to the eye in the form of anophthalmic emulsion, solution, ointment, gel, or eye drop. The ocular tissue injuries include but are not limited to chemical, thermal, and mechanical tissue injuries including abrasion, penetrating injuries, scrapes, scratches, cuts, and punctures, as well as biological injuries, radiological injuries, nuclear injuries, injuries induced by irritating agents, eye infections, ocular surface diseases, corneal dystrophies, corneal ectasia, intraocular inflammation (uveitis), retinal diseases, glaucoma, open- and closed-globe trauma, and the like. The SSOE is packaged into a ready-to-use canister or blister pack and configured to be directly applied to the eye without the need for additional manipulation. The SSOE, which maintains its oxygen-releasing capacity in room temperature storage up to one year, remains on/in the injured tissues and continues to release oxygen over hours, reducing the need for repeated application. It is compatible with ocular surface cells and shows no toxicity to live animals. Most importantly, a single topical application of SSOE significantly reduces disease burden after injury. Therefore, the PFD-based supersaturated oxygen emulsion (SSOE) of embodiments is a safe, effective, and ready-to-use therapeutic in treating acute ocular injury. Thus, a single topical application of SSOE after injury reduces tissue hypoxia, apoptosis, and inflammation, leading to preserved tissue integrity and reduced cataract formation and optical opacification.

The SSOE of an embodiment comprises but is not limited to water (approximately 68.20% w/w (i.e., percentage weight of PFD of total weight of SSOE)), Phospholipon 90H (approximately 5.00 w/w), Polawax (approximately 1.80% w/w), and Rejuvenox (approximately 25.00% w/w). The Rejuvenox comprises PFD. The SSOE of an alternative embodiment comprises but is not limited to water (approximately 24.70% w/w), white petrolatum (approximately 12.00% w/w), Phospholipon 90H (approximately 6.00% w/w), Polawax (approximately 1.80% w/w), Rejuvenox (approximately 55.00% w/w), and vitamin E acetate USP (approximately 0.50% w/w).

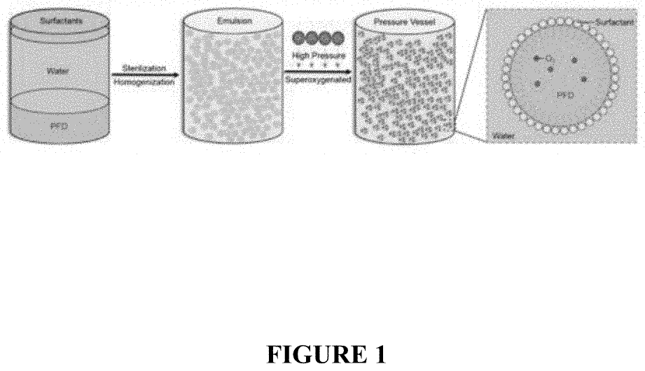

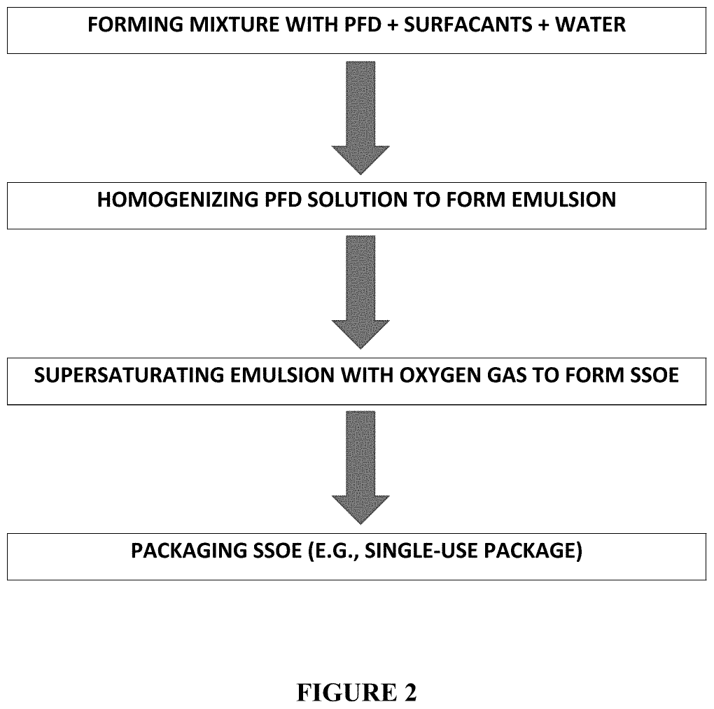

FIG. 1 is a flow diagram of a method of forming the supersaturated oxygen emulsion (SSOE), under an embodiment. Approximately 25% w/v PFD (i.e., percentage weight of PFD of total volume of SSOE) was homogenized with Phospholipon 90H, Polawax, and water; the emulsified nanoparticles were then supersaturated with medical grade oxygen gas in a high-pressure chamber. The oxygenated emulsion has a more uniform and reflective appearance when compared with non-oxygenated emulsion. The final SSOE was packaged in small pressurized dispensing canisters. FIG. 2 is a flow diagram of a method of forming and packaging the SSOE, under an embodiment. The structure of SSOE particles was observed under transmission electron microscopy and the diameter of most particles was between 50 and 300 nm. By maintaining pressure within the canister, dissolution and outgassing are prevented or at least mitigated during storage so that high oxygen concentration will be delivered on dispensation.

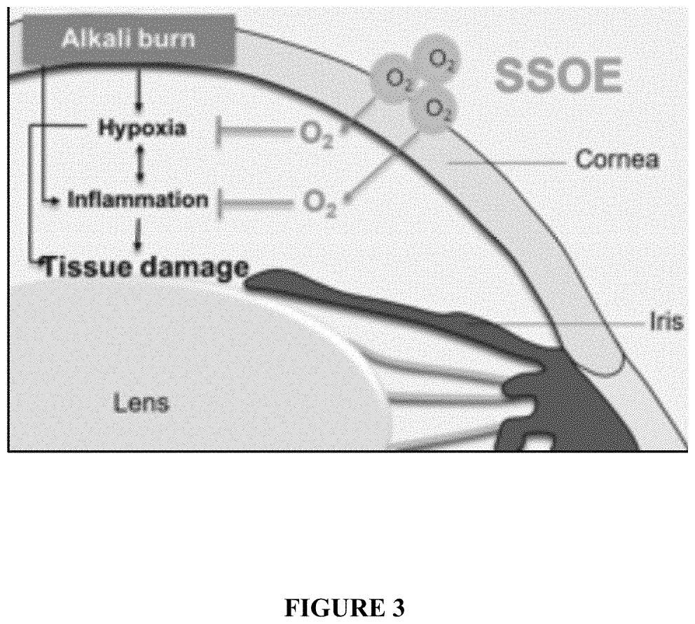

FIG. 3 shows a method of treating an ocular injury (e.g., alkali burn) by applying the SSOE, under an embodiment. The SSOE herein can be directly dispensed from the canister without additional equipment or manipulation. In addition, disposable plastic pipettes can be used to transfer the emulsion after dispensation. The pH of the emulsion is approximately 6.7, within the safety range of ophthalmic use. Upon dispensation, the partial pressure of oxygen (pO2) within the emulsion reached a peak of approximately 600 mmHg in a few minutes (approximately four times the atmospheric pO2 (160 mmHg) and remained above 600 mmHg for at least 2.5 hours. The unopened packaged emulsion was stored in a normal environment at room temperature. The pO2 of the stored emulsion decreased slightly compared to freshly prepared emulsion, but the pO2 within the emulsion remained above 500 mmHg when dispensed after one (1) year of storage. The inherent viscosity of the SSOE described herein resulted in its remaining on the ocular surface after application.

The biocompatibility of the SSOE described herein with cultured human corneal cells and its safety on intact eyes was demonstrated. Results showed SSOE treatment of embodiments drastically prevented the loss of optical transparency, significantly accelerated epithelial wound healing, and mitigated tissue fibrosis and scar formation.

In untreated eye tissue, gradual clouding of the crystalline lens (cataract formation) was observed after injury, and the rate of visually significant cataract formation eyes was significantly higher than the rate in SSOE-treated eyes. Further, the majority of SSOE treated ocular tissue had no signs of cataract formation while being studies.

The anatomy of the anterior segment of the eye generally includes the cornea, iris, lens, and the anterior chamber (formed between the cornea and iris/lens). Treatment with the SSOE of embodiments reduced exudation and maintained the anterior chamber depth (approximately 300 μm) of the eye close to a normal typical depth. Further, the angle of the anterior chamber, where the corneal endothelium and the root of iris meet, was maintained in SSOE-treated eyes.

SSOE treatment of ocular injury mitigated ocular hypoxia in the anterior chamber and by producing a rapid and robust increase in anterior chamber oxygen concentration within in a few seconds of application, and maintaining the increase in oxygen concentration above baseline level for more than 60 minutes.

Hypoxia leads to cell apoptosis by inducing mitochondrial damage, and increased oxygen free radicals. Cell apoptosis was examined using Terminal deoxynucleotidyl transferase dUTP nick-end labeling (TUNEL) staining at one and four hours after injury. At one hour after burn, nearly all cells in the central corneas of untreated or vehicle-treated eye were TUNEL-positive. In SSOE treated eyes, the TUNEL positive cells were seen mostly at the superficial epithelial layer, which was in direct contact with the injury agent. Less TUNEL staining was observed in the iris of the SSOE-treated eyes, compared with untreated and vehicle-treated eyes. Interestingly as the impact of tissue injury propagated beyond the cornea post-burn, the lens epithelial cells lining up the anterior lens capsule underwent apoptosis in untreated eyes; whereas there was little TUNEL positivity in the lens of SSOE-treated eyes. TUNEL positive cells were not observed in the retina or optic nerve at these time-points.

Tissue inflammation was also examined. Ocular tissue injury resulted in the production of inflammatory cytokine IL-1β and an increase in the expression of inflammatory mediator MMP9, while SSOE treatment decreased their levels in both the cornea and iris/ciliary body. In addition, the injury led to massive infiltration of leukocytes from the conjunctiva to the limbus and peripheral cornea (shown with the use of CD45 immunostaining). Treatment with the SSOE of embodiments led to less infiltration compared to untreated eyes. Consistent with immunohistochemical staining, a much higher percentage of CD45-positive cells in the conjunctiva in the untreated eyes was observed compared to SSOE-treated eyes.

More particularly, the biocompatibility of the SSOE with cultured human corneal cells was demonstrated in vitro as described herein, and its safety on intact mouse eyes in vivo was evaluated using a modified Draize test. To evaluate its efficacy in treating acute burns, corneal burns were induced using sodium hydroxide (NaOH), followed by copious irrigation in adult BALB/c mice. SSOE or un-oxygenated vehicle control (Vehicle) were then immediately applied to the ocular surface for 40 minutes while mice were under anesthesia. The emulsion (SSOE or vehicle) was then washed off and antibiotic eye ointment was placed in all groups. These mice received no additional treatment afterward. Mice that received ocular burns but not SSOE or vehicle treatment were also included as untreated controls. The ocular surface and anterior segment changes were evaluated with slit lamp photography at 24 hours, 48 hours, 5, 7, 14, 21, and 28 days.

After alkali burn, untreated and vehicle-treated eyes developed progressive and significant optical opacities, a combination of corneal opacification and cataract formation. SSOE treatment of embodiments herein drastically prevented the loss of optical transparency, resulting in significantly lower opacity scores as early as the second day after burn and its protective effect lasted up to 1 month (p<0.0001).

The formation of new blood vessels (neovascularization) in the normally avascular corneas was examined. Corneal neovascularization began at the limbus as early as two days after burn and extended to the center on Days 21 to 28. SSOE treatment resulted in a small and statistically non-significant decrease in neovascularization score at Days 21 and 28. The centricity of these vessels (length of the longest vessel from the limbus) was comparable among all three (3) groups at all time points, indicating that SSOE treatment had limited effect in reducing corneal neovascularization.

In the untreated and vehicle-treated eyes, serial slit lamp photography demonstrated the gradual clouding of the crystalline lens (cataract formation) after burn. By Day 7, the rates of visually significant cataract in the untreated and vehicle-treated groups were approximately 83.3% and 92.0%, respectively, significantly higher than the rate of 19.0% in SSOE-treated eyes (p<0.0001). Of the 21 SSOE-treated eyes, 17 had no signs of cataract formation throughout the 28-day study duration.

In the immediate post-burn period, untreated and vehicle-treated eyes had significant corneal epithelial defects, evidence by fluorescein staining. SSOE treatment of embodiments significantly accelerated epithelial wound healing at 24 hours (p=0.0003, compared with untreated group; p=0.0122, compared with vehicle control group). Corneal epithelial defects healed in all three groups by 48 hours, but scattered superficial punctate staining was present, indicating that epithelial barrier function (tight junctions) had not restored. SSOE treatment resulted in less punctate staining at 48 hours, compared with untreated and vehicle groups. On day five after burn, there was no staining in any of the three groups.

To investigate the effect of SSOE on ocular micro-anatomy and tissue integrity after alkali burn, serial optical coherence tomography (OCT) imaging was performed in live mice. The anatomy of the anterior segment of the eye generally includes the cornea, iris, lens, and the anterior chamber (formed between the cornea and iris/lens). Alkali burn resulted in persistent corneal edema, evidenced by increased corneal thickness and neither vehicle nor SSOE treatment had significant effects on corneal edema at any time point. Alkali burn led to exudation within the anterior chamber of the eye, followed by subsequent narrowing of the anterior chamber, and in severe cases adhesion of the iris/lens to the cornea (anterior synechiae). SSOE, but not vehicle treatment, resulted in much reduced exudation and maintained the anterior chamber depth (approximately 300 μm) close to that of normal mice (350 μm).

Hematoxylin and eosin (H&E) staining on Day 28 after chemical burn confirmed OCT findings. In the untreated and vehicle-treated eyes, there was massive leukocyte infiltration, tissue fibrosis, and distortion within the cornea, as well as significant exudation within the anterior chamber and adhesion of the iris/lens to the cornea; whereas in SSOE-treated corneas, corneal infiltration was more consolidated, and the anterior chamber was maintained. The angle of the anterior chamber, where the corneal endothelium and the root of iris meet, is obliterated in untreated and vehicle-treated eyes; whereas it was maintained in the SSOE-treated ones.

A marker for myofibroblasts, α-smooth muscle actin (αSMA), highlights tissue fibrosis and scar formation. The anterior segment of the eye including the cornea is void of αSMA staining in normal condition. In untreated and vehicle-treated eyes after alkali burn, there was intense αSMA staining throughout the entire cornea and in part of the iris. SSOE treatment on the other hand resulted in scarce αSMA staining.

Consistent with previous reports that ocular alkali injury led to ocular hypoxia, using a micro-oxygen sensor, a 60% decrease in oxygen concentration within the anterior chamber seconds after burn was demonstrated (165.4±20.3 μmol/L to 66.1±15.6 μmol/L, p<0.0001). Using this micro-oxygen sensor to record in real time, a sharp decline in oxygen level after burn was observed and the level gradually returned to normal range in 50 minutes. While in vehicle-treated eyes, anterior chamber oxygen concentration remained below baseline level up to 60 minutes, SSOE treatment led to a rapid and robust increase in oxygen concentration, peak level 534.8±164.7 μmol/L in a few seconds, and such increase maintained above baseline level for more than 60 minutes.

Hypoxia leads to cell apoptosis by inducing mitochondrial damage, and increased oxygen free radicals. Cell apoptosis was examined using Terminal deoxynucleotidyl transferase dUTP nick-end labeling (TUNEL) staining at one and four hours after alkali injury. At one hour after burn, nearly all cells in the central corneas of untreated or vehicle-treated eye were TUNEL-positive. In SSOE treated eyes, the TUNEL positive cells were seen mostly at the superficial epithelial layer, which was in direct contact with NaOH. Less TUNEL staining was observed in the iris of the SSOE-treated eyes, compared with untreated and vehicle-treated eyes. Interestingly as the impact of alkali injury propagated beyond the cornea at four hours post-burn, the lens epithelial cells lining up the anterior lens capsule underwent apoptosis in untreated and vehicle-treated eyes; whereas there was little TUNEL positivity in the lens of SSOE-treated eyes. TUNEL positive cells were not observed in the retina or optic nerve at these time-points.

Tissue inflammation was also examined. Alkali burn resulted in the production of inflammatory cytokine IL-1β and an increase in the expression of inflammatory mediator MMP9. SSOE, but not vehicle treatment, decreased their levels in both the cornea and iris/ciliary body. In addition, alkali burn led to massive infiltration of leukocytes from the conjunctiva to the limbus and peripheral cornea 24 hours after the burn (shown with the use of CD45 immunostaining). Treatment with the SSOE of embodiments led to less infiltration compared to untreated and vehicle-treated eyes. Consistent with immunohistochemical staining, a much higher percentage of CD45-positive cells in the conjunctiva in the untreated (29.7±7.0%) and vehicle-treated (28.3±5.3%) eyes was observed, compared to SSOE-treated ones (18.4±5.2%) using flow cytometry.

Methods used in developing results described herein used BALB/c mice obtained from Charles River Laboratories (Wilmington, Mass.) and housed in the animal vivarium of the Schepens Eye Research Institute. All animal procedures were performed in accordance with the Association for Research in Vision and Ophthalmology (ARVO) statement for the use of Animals in Ophthalmic and Vision Research and approved by the Animal Care Committee of the Schepens Eye Research Institute. Preliminary study used equal numbers of male and female adult mice and showed no sex-based differences in clinical efficacy. Adult male mice between the ages of 8 and 10 weeks were used.

SSOE emulsion was applied onto carbon and formvar coated 200 mesh copper grids and air dried. Grids were imaged using a FEI Tecnai G2 Spirit transmission electron microscope (FEI, Hillsboro, Oreg.) at 100 kV interfaced with an AMT XR41 digital CCD camera (Advanced Microscopy Techniques, Woburn, Mass.).

Partial oxygen pressure (pO2) in SSOE and cell culture media was measured with DP-PSt7-10 oxygen sensor (PreSens, Regensburg, Germany). In brief, 1 ml SSOE was dispensed into a 3.8 cm-sq. culture plate. The sensor was immediately immersed within the emulsion or culture media and the pO2 values were recorded in real time. The anterior chamber oxygen measurement was performed using the DP-PSt7-2 oxygen sensor. Briefly, anesthetized mice were mounted in a stereotaxic frame and the oxygen sensor was immobilized on a manual micromanipulator. A 28-gauge needle was used to perform a narrow, self-sealing tunnel in the temporal region of the cornea, adjacent to the limbus. Before the insertion of the needle tip, it was marked with surgical dye (Accu-line Products Inc., Hyannis, Mass.) to guide the insertion of the sensor probe into the anterior chamber.

Mice were anesthetized using 60 mg/kg ketamine and 6 mg/kg xylazine, and deep anesthesia was confirmed by a toe pinch test. One drop of 0.5% Proparacaine hydrochloride USP (Bausch &Lomb, Tampa, Fla.) was applied to the right eye for 1 minute. A sterile 2-mm-diameter filter paper disc (1001-329, Whatman, Maidstone, UK) was soaked in 1 mol/L sodium hydroxide solution for 10 seconds. Excess sodium hydroxide was dried by touching the edge of the filter paper disc on a paper towel once. The filter paper disc was then applied onto the central cornea (sparing limbus) for 20 seconds. After removing the filter paper, the ocular surface was irrigated with PBS via an 18-gauge needle attached to an intravenous infusion bag until the pH level of the ocular surface returned to 7. Mice were then placed on a heating pad, and SSOE or vehicle control emulsion (approximately 50 μl) was applied on the ocular surface of the burned eye for 40 minutes while the mice were still under anesthesia. After the mice woke up, 0.05 mg/kg Buprenorphine hydrochloride (Reckitt Benckiser Healthcare Ltd, Hull, UK) was administered by subcutaneous injection for pain management and triple antibiotic eye ointment (Bausch & Lomb, Tampa, Fla.) was applied topically to prevent infection. No additional ocular treatment was given afterwards.

Corneal fluorescein staining was used to evaluate corneal epithelial damage. Briefly, 1 μl of 2% fluorescein (Sigma-Aldrich, St. Louis, Mo.) was applied into the lateral conjunctival sac of the mice for 10 seconds and then the eye was washed with PBS to remove the excessive fluorescence. The corneas were immediately examined with a slit lamp biomicroscope (Topcon, Tokyo, Japan) under cobalt blue light. Corneal epithelial defects were determined by measuring the stained areas. Corneal epithelial punctate staining was scored using the standard National Eye Institute grading system of 0 to 3 for each of the five areas of the cornea (central, superior, inferior, nasal and temporal) and then totaled (score range 0-15). Optical opacity was scored in 0 to 4. Cornea neovascularization was assessed using a modified scoring system. A score of 0-4 was assigned to each of the five areas of the cornea (central, superior, inferior, nasal and temporal), based on the area occupied by new blood vessels: 0, no new blood vessels; 1, less than 30% area; 2, more than 30% area but less than 70%; 3, more than 70% area less than 100%; and 4, 100% area (range 0-20). Anterior segment images were taken using anterior segment-optical coherence tomography (OCT) Bioptigen Spectral Domain Ophthalmic Imaging System Envisu 82200 with 12 mm telecentric lens (Bioptigen Inc, Durham, N.C.). Corneal thickness and anterior chamber depth were measured using the OCT built-in software.

Mouse whole eyes were sectioned (9 μm in thickness) and fixed in 4% paraformaldehyde for 20 min. The slides were incubated in 0.2% Triton X-100 for 20 min and then 2% BSA for 1 h at room temperature. Slides were incubated with anti-αSMA (14-6496-82, eBioscience, San Diego, Calif.) and anti-CD45 (103101, Biolegend, San Diego, Calif.) antibodies at 4° C. overnight, followed by Alexa Fluor 488-conjugated donkey anti-mouse or rat secondary antibody (Invitrogen, Carlsbad, Calif.) for 1 h at room temperature. The slides were mounted with DAPI mounting medium (H-1200, Vector lab, Burlingame, Calif.) and photographed under confocal laser scanning microscope (SP5, Leica, Wetzlar, Germany).

Primary human corneal epithelial cells (hCECs) were isolated from donor corneas (Eye Bank Association of America, Washington, D.C.) and cultured in Corneal Epithelial Cell Complete Medium (ATCC, Manassas, Va.). Human corneal fibroblast cells (hCFCs) were from ATCC and cultured in DMEM/F12 basal medium (Thermo Fisher Scientific, Waltham, Mass.) comprising approximately 10% v/v FBS (i.e., percentage volume of FBS of total volume), approximately 1% v/v antibiotic/antimycotics (Sigma-Aldrich), and approximately 1% (v/v) L-glutamine (Sigma-Aldrich). Human corneal endothelial cell line (hCEnC-21T) (Schepens Eye research Institute) was cultured in a supplemented Chen medium (OptiMEM-I, Invitrogen). All cells were cultured at 37° C. in a 5% CO2 incubator. To determine the biocompatibility of SSOE with cultured cells, hCECs were grown to 100% confluence and the completed media was replaced with equal volume of SSOE, PBS, or vehicle control for one hour. hCFCs and hCEnCs were grown to confluence and 2 ml of SSOE, PBS, or vehicle control was placed in a transwell insert, which was then placed in the culture dish for one hour.

Cell viability was evaluated by using LIVE/DEAD™ Viability/Cytotoxicity Kit (L3224, Invitrogen). Briefly, cells were incubated for 30 minutes at room temperature with a mixture of 2 μM calcein acetoxymethyl ester (Calcein AM) and 4 μM ethidium homodimer-1 (EthD-1). After one wash with 1×PBS containing 1 ug/ml Hoechst 33342 (H3570, Invitrogen) to visualize the nucleus, the live cells (green fluorescence) and dead cells (red fluorescence) were photographed using fluorescence microscope (DMi8, Leica).

In situ cell apoptosis was determined by TUNEL assay (Roche, Basel, Switzerland) in frozen sections of whole eyes. Sections were counterstained with DAPI and images were taken with the confocal laser scanning microscope (SP8, Leica). DAPI signal (blue) was overlaid with Texas red (TUNEL positive cells) and quantified using ImageJ software. TUNEL positivity was calculated as the ratio of TUNEL-positive cells/DAPI-positive cells (%). For each group, at least three individual mice were included.

Whole eyes were harvested and fixed in 4% paraformaldehyde overnight. Sections having 6-μm thickness were stained with hematoxylin and eosin and examined under a light microscope (DMiL, Leica).

Corneal and iris/ciliary body tissues were harvested under dissecting microscope and placed in TRIzol solution (Ser. No. 15/596,026, Invitrogen). Total RNA was isolated with Direct-zol RNA Kit (R2060, Zymo research, Irvine, Calif.) following the manufacturer's protocol and reverse transcribe to cDNA with RevertAid H Minus Reverse Transcriptase (EP0452, Thermo Fisher Scientific). Real-time PCR was performed using a SYBR Green Master Mix Kit (A25742, Thermo Fisher Scientific) on a StepOne plus Real-Time PCR System (Applied Biosystems, Foster City, Calif.). The cycling conditions for reactions are 40 cycles of 95° C. for 15 s, 60° C. for 60 s. The mouse IL-1β, MMP9 and β-actin gene primers were designed using the Primer 3 system and their sequences are showed as follows: β-actin, 5-cctaaggccaaccgtgaaaag-3, 5-aggcatacagggacagcacag-3; IL-1β, 5-tggaccttccaggatgaggaca-3, 5-gttcatctcggagcctgtagtg-3; MMP9, 5-ctgactacgataaggacggca-3, 5-tagtggtgcaggcagagtagga-3. The results of the relative qRT-PCR were analyzed by the comparative threshold cycle (CT) method and normalized to β-actin expression as the reference gene.

The conjunctival tissue was separated and subsequently digested in DMEM media containing 2 mg/mL collagenase D (11088866001, Roche, Indianapolis, Ind.) and 0.2 mg/mL DNase I (10104159001, Roche) for 2 hours at 37° C. Tissue suspension was filtered through a 70 μm cell strainer (BD Falcon; Becton-Dickinson, Franklin Lakes, N.J.). After washing with 5% FBS, single cells in suspension were incubated with Fc blocking antibody at 4° C. for 30 min. Cells were then immunostained with the APC/Cy7-conjugated anti-CD45 antibody (103116, Biolegend), or isotype-matched control antibody (400624, Biolegend). The stained cells were analyzed using the LSRII flow cytometer (BD Biosciences, San Jose, Calif.) and FCS Express software (De Novo Software, Los Angeles, Calif.).

The statistical analysis was conducted using GraphPad Prism software (GraphPad Software Inc., San Diego, Calif.). All summary data are reported as means±SEM. Comparisons between 2 groups were performed by an unpaired, two-tailed Student's t-test; and comparison among 3 groups were performed by One-way ANOVA test. The rate of cataract formation between groups was compared by Fisher's exact test. P<0.05 was considered statistically significant.

In Vitro Study of SSOE In Primary Human Corneal Epithelial Cells

Further study involving topical treatment of ocular trauma with the SSOE of an embodiment involved in vitro studies carried out in primary human corneal epithelial (HCE) cells, as the outermost layer is the first barrier to chemical and mechanical injury. Chloropicrin (CP) was used as a chemical agent to induce ocular injury in HCE cells. CP was used to induce injury because of its irritating, choking and powerful lacrimating tear gas like properties. The breakdown of products of CP, which is currently used as a fungicide, herbicide, insecticide, and nematicide in agriculture, include chlorine and phosgene, which could also contribute to the toxicity from its exposure. Due to strong irritant and toxic properties, no available antidote, and high potential to induce ocular and other tissue injuries, CP poses a potent threat for accidental, occupational, intentional and battlefield exposures. CP is absorbed into the cornea and conjunctiva, causing ocular irritation, lacrimation, intense pain, and burning. This can lead to corneal injury with epithelial and stromal necrosis, edema, neovascularization, inflammation, and corneal endothelial loss.

Resulting data indicate that the SSOE releases oxygen in a time-dependent manner, reaching a partial pressure within the emulsion over four times atmospheric levels. Studies in HCE cells indicate SSOE is safe when diluted in culture media and applied topically to HCE cells. Additionally, the SSOE may promote wound closure and enhance cellular viability. SSOE treatment is effective in reversing the chemical agent-induced increases in markers of DNA damage and apoptotic cell death in HCE cells. The application of SSOE therefore was shown not to lead to DNA damage, promote cell death, or hinder the rate of scratch closure and enhances cellular viability. Consequently, maintaining adequate tissue oxygenation is critical for tissue preservation and wound repair, especially in avascular tissues like the cornea.

As described above, chloropicrin (CP, PS, CCl₃NO₂, nitrochloroform, trichloronitromethane), an aliphatic nitrate compound, was used as a model agent to induce chemical injury in human corneal epithelial (HCE) cells. CP is a colorless liquid with irritating, choking, and lacrimating properties and exposure primarily affects the eyes, skin, and respiratory system. CP ocular exposure causes eye irritation, associated with lacrimation and inflammation, which involves corneal edema, ocular tissue damage, and visual damage. The cornea is the outermost layer of the eye and is highly sensitive to chemical exposures and injuries; thus, studies were conducted using HCE cells. The effect of SSOE treatment on CP-induced toxicity in HCE cells was investigated. Results of an embodiment indicate SSOE of embodiments herein is safe to use on HCE cells and improves wound healing, as assessed via in vitro assays.

The chemicals and reagents of an embodiment include chloropicrin, 3-(4,5-dimethylthiazol-2-yl)-2,5-diphenyltetrazolium bromide (MTT) and anti-beta-actin antibody obtained from Sigma-Aldrich (St. Louis, Mo.). Primary HCE cells and culture media were obtained from ATCC (Manassas, Va.), and TrypLE Express was from Thermo-Fisher Scientific (Waltham, Mass.). Primary antibodies for phosphorylated H2A.X (Ser139), phosphorylated p53 (Ser15), p53, cleaved-poly (ADP-ribose) polymerase (PARP), and anti-mouse and anti-rabbit IgG horseradish peroxidase (HRP)-conjugated secondary antibodies were obtained from Cell Signaling Technology (Beverly, Mass.). The detergent compatible protein assay kit was purchased from Bio-Rad Laboratories (Hercules, Calif.). Enhanced chemiluminescence kit (ECL) was from GE healthcare Bio-Sciences (Pittsburgh, Pa.). For scratch wound assay, 35-mm culture-insert μ-dish was purchased from ibidi (ibidi USA, Fitchburg, Wis.). Cyclooxygenase-2 (COX-2) antibody was from Cayman chemicals (Ann Arbor, Mich.).

Unoxygenated and oxygenated (30% and 55% w/v PFD) formulations of the SSOE were manufactured by Suite-k (Edison, N.J.). The pO2 in each sample of SSOE (unoxygenated and oxygenated, 30% and 55% w/v PFD) was evaluated using the Microx 4 oxygen meter and oxygen dipping probe (PreSens Precision Sensing GmbH, Regensburg, Germany) according to manufacturer's instructions.

Primary HCE cells (ATCC, Manassas, Va.) were grown in corneal epithelial cells basal medium (ATCC) supplemented with corneal epithelial cell growth kit (ATCC) under standard cell culture conditions. CP Exposure and SSOE Treatment HCE cells were cultured in 100-mm culture plates. At 60 to 70% confluency, media was removed and the cells were either exposed to CP (50 μM CP for 30 minutes) or left unexposed (Control). After the exposures, the cells were washed and either fresh media or media containing 0.25 g/mL of SSOE (55% PFD) were added and cells were cultured for 24 hours. After 24 hours of exposure, cell lysates were prepared and analyzed by Western immunoblotting as described previously. Briefly, after SDS-PAGE, samples were transferred to nitrocellulose membrane. Upon blocking with nonfat dry milk, membranes were probed with appropriate primary antibodies (overnight at 4° C.) followed by incubation with peroxidase-conjugated secondary antibody. Protein loading was confirmed by stripping and re-probing the membranes with β-actin antibody.

The cell viability (MTT) assay involved HCE cells (5×10e4 cells/well) seeded in 24-well plates and grown overnight under standard culture conditions. The following day, culture media were changed, and the cells were cultured with either 1 mL of media only (Control) or media containing 0.0625 or 0.25 g/mL of the unoxygenated vehicle or media containing 0.0625 or 0.25 g/mL of the SSOE with either 30% or 55% PFD (6 wells/each) for 24 hours. Culture media were then removed, and the cells were incubated with 0.5 mg/mL of MTT (Sigma-Aldrich St. Louis., Mo.) for four hours at 37° C. After removing MTT solution, dimethyl sulfoxide was added to the wells and absorbance was read at 540 nm using Spectra max 190 micro plate reader (Molecular Devices, Sunnyvale, Calif.).

Scratch wound assay involved HCE cells (70 μL of 5×10e6 cells/mL) seeded in 35-mm culture-insert μ-dish (ibidi USA, Fitchburg, Wis.). After overnight incubation, the culture inserts were removed. Images were taken (0 hour) and the plates were then cultured with either 1 mL of media only (Control), media containing 0.0625 or 0.25 g/mL of the unoxygenated vehicle, or media containing 0.0625 or 0.25 g/mL of the SSOE (55% PFD) in triplicate. At two and four hours after the addition of the emulsions, media from each well were aliquoted to a 24-well plate. After removing the media, the wells were washed twice, fresh media were added, and images were taken. After imaging, the aliquoted media were added back to the wells. The images were analyzed using ImageJ software and the percent increase in total cell number or area at two or four hours was calculated for each plate (n=3).

Western blot analyses involved cell lysates prepared after the CP exposure and treatments in HCE cells, and protein estimation was carried out. About 60 μg of the samples were subjected to SDS-PAGE as described earlier, and Western blot analyses were carried out as described previously. Briefly, after SDS-PAGE, samples were transferred to nitrocellulose membrane. Upon blocking with nonfat dry milk, membranes were probed with appropriate primary antibodies (overnight at 4° C.) followed by incubation with peroxidase-conjugated secondary antibody. Protein loading was confirmed by stripping and re-probing the membranes with β-actin antibody.

Data were analyzed using a one-way analysis of variance with Tukey or Bonferroni t-test for multiple comparisons (Sigma Stat 2.03). Differences were considered significant for P values<0.05. Data are presented as the mean+/−standard error of mean (SEM; n=3).

Regarding SSOE oxygen concentration, results of an embodiment showed the pO2 in each sample of SSOE (unoxygenated and oxygenated, 30% and 55% w/v PFD) was evaluated using the Microx 4 oxygen meter and oxygen dipping probe (PreSens Precision Sensing GmbH, Regensburg, Germany). A time-dependent test of oxygen release was conducted using a small (golf ball-sized) bolus of the 55% PFD formulation. Immediately after being dispensed, the pO2 in the SSOE rose to over 550 mm Hg (Torr) and remained over 500 mm Hg for longer than 22 hours.

The effect of SSOE treatment on HCE cell viability was assessed via MTT assay and results showed SSOE increased HCE cell viability. Following SSOE exposure, there was an observed increase in HCE cell proliferation. Cell viability in the 0.0625 and 0.25 g/mL SOE-treated groups (55% PFD) increased significantly as compared to Control. Cell viability increased by 1.5- and 1.4-fold in the 0.0625 and 0.25 g/mL SOE-treated group (55% PFD), respectively, in comparison with untreated cells (Control).

Results of embodiments described herein also showed SSOE promotes wound healing in cultured HCE cells. The SSOE-induced increase in cell viability could also be accompanied with an increase in cell proliferation. SSOE exposure promoted wound healing (closure of the scratch) in HCE cells, which was investigated via wound scratch assay. After two-hour incubation with the SSOE, a 1.4-fold increase in cell coverage (area) was observed for both concentrations of exposure (0.0625 and 0.25 g/mL) as compared to the Control. After four hours of SSOE exposure, a 1.6- and 1.3-fold increase in cell coverage in the 0.0625 and 0.25 g/mL groups, respectively, was observed compared to Control.

SSOE treatment of embodiments was shown to reverse CP-induced markers of DNA damage and apoptosis in HCE cells. Treatment with SSOE (55% PFD, 0.25 g/mL) did not lead to the expression of markers of DNA damage or apoptotic cell death in HCE cells. There was no induction of H2A.X (Ser139), a marker for double-stranded DNA breaks; p53 (Ser15), a key molecule involved in DNA repair; and PARP, the executor of apoptosis. However, an increase in COX-2 expression was observed. Following CP exposure, HCE cells showed enhanced expression of proteins related to DNA damage and cell death, as indicated by a strong increase in the phosphorylation of H2A.X (Ser139), p53 (Ser15), and accumulation of cleaved-PARP. Treatment with SSOE (55% PFD, 0.25 g/mL) led to a complete reversal in H2A.X (Ser139) and p53 (Ser15) phosphorylation, as well as cleaved-PARP. However, as SSOE exposure alone led to increased COX-2 expression, no change in COX-2 levels was observed when SSOE was added after CP exposure. In fact, the increase in COX-2 expression following SSOE alone was higher than that following CP alone.

Embodiments therefore tested the effects of SSOE treatment on HCE cells, as corneal epithelial cells are most sensitive to chemical and mechanical injuries. Analyses carried out with the SSOE showed an increase in cell viability indicating that SSOE treatments do not diminish HCE cell viability but rather have proliferative effects on HCE cells. An increase in wound closure was also observed upon SSOE treatment in HCE cells, providing further evidence in support of the cellular growth promoting ability of the SSOE. Embodiments showed SSOE treatment alone does not cause DNA damage and does not induce apoptotic cell death and oxidative stress markers. CP exposure caused an increase in DNA damage and apoptotic cell death marker in HCE cells, however the CP-induced increase in markers of DNA damage and apoptotic cell death were abrogated upon SSOE treatment.

Ex Vivo Study of SSOE in Rabbit Corneas

Following on the results described herein in which human corneal epithelial cells showed the treatment potential of SSOE (55%), SSOE efficacy was further tested in an ex vivo CP-induced rabbit corneal injury model. Generally, corneas were exposed to CP (700 nmol) for two hours and washed and cultured with or without SSOE for 24 hours or 96 hours. At 96 hours post CP exposure, SSOE treatment enhanced healing of the corneal epithelial layer, and abrogated the CP-induced epithelial cell and keratocyte apoptotic death. SSOE treatment also reversed the CP induced DNA damage (H2A.X phosphorylation) and inflammatory markers (e.g. MMP9, IL-21, MIP-1β, TNFα).

As described herein, CP was used to induce injury because it is absorbed into the cornea and conjunctiva, causing ocular irritation, lacrimation, intense pain, and burning, which can lead to corneal injury with epithelial and stromal necrosis, edema, neovascularization, inflammation, and corneal endothelial loss. Since the cornea is highly sensitive to CP exposure, the studies described herein in human corneal epithelial (HCE) cells have shown that oxidative stress plays a role in CP-induced DNA damage, lipid peroxidation, and protein carbonylation, leading to its toxicity. In addition, corneal wounds from chemical exposures are often hypoxic, leading to tissue damage.

Oxygen treatment is effective in ocular injuries and in patients with ocular chemical burns as described herein. Oxygen therapy improves limbal ischemia, promote epithelialization and corneal transparency, and reduces corneal vascularization in ocular injury. Further, results of studies involving the PFD-based SSOE of embodiments described herein indicate SSOE (55%) application increased cell viability and wound healing, and its therapeutic potential in mitigating CP-induced DNA damage, apoptotic cell death, and oxidative stress markers in HCE cells.

More particularly, studies of SSOE embodiments described herein included an ex vivo rabbit corneal organ culture model with CP in order to further study the mechanisms underlying the CP-induced corneal pathology and establish biomarkers for efficacy studies with SSOE and other treatments. This CP-induced ex vivo corneal injury model enabled further evaluation of the therapeutic potential of SSOE in reversing histopathological and molecular markers of CP-induced corneal injury and inflammation.

Materials and methods of embodiments herein include chemicals and reagents comprising CP, ascorbic acid, ciprofloxacin, and 1×RPMI 1640 vitamin solution obtained from Sigma-Aldrich Chemical Co. (St. Louis, Mo.). Dulbecco's modified eagle's medium (DMEM) and 100×minimum essential medium-non-essential amino acids (MEM-NEAA) were obtained from Gibco (Life technologies, NY). SSOE vehicle (unoxygenated emulsion) and oxygenated (55% w/v PFD) SSOE were obtained from Coruna Medical LLC. H2AX (Ser 139) antibody was obtained from Abcam Inc. (Cambridge, Mass.) (Microx 4 Oxygen Meter, PreSens, Germany). Apoptosis detection kit (DeadEnd Colorimetric TUNEL System) was obtained from Promega (Madison, Wis.). Cytokine array kit (Quantibody® Rabbit Cytokine Array 1) was obtained from RayBiotech (Norcross, Ga.).

Rabbit corneal organ culture and CP exposure included rabbit eyes (male, New Zealand white, 8-12 weeks) purchased from Pel-Freez (Pel-Freez Biologicals, AR) and shipped overnight in DMEM and antibiotics. Corneas were dissected with scleral rim and cultured overnight in a 12-well plate in DMEM (containing 1×MEM NEAA, 1× RPMI 1640 vitamins, 0.1 mg/ml ascorbic acid and 0.01 mg/ml ciprofloaxin) at 37° C. in a humidified 5% CO2 incubator. Corneal injury was induced by moistening the anterior corneal surface with DMEM medium and then exposing it to 700 nmol CP (10 μL in the growth media drop wise on the center of cornea; CP stock was prepared in DMSO and diluted in media for the desired concentration) for two hours. Thereafter, the corneas were washed (three times with media) and were further cultured for 24 hours or 96 hours. Following the organ culture duration, the corneas were equally divided into four sections and one quarter was fixed in 10% phosphate-buffered formalin, and the remaining sections were snap frozen in liquid nitrogen for further analyses.

To measure the oxygen penetration, the oxygen sensor probe (Microx 4 Oxygen Meter, PreSens, Germany) was inserted through a needle hole at the limbus and the tip placed just under the endothelial cell layer of the cornea in the whole ex vivo rabbit eyes. The eye was allowed to equilibrate for a few hours, and then the SSOE (55% w/v PFD) was placed directly around the cornea (about 5 mm thick). Recording of the oxygen levels proceeded overnight at room temperature.

For SSOE treatments, following two hours of CP exposure, corneas were washed and either treated with SSOE (55% w/v PFD) or left untreated and further cultured till 24 hours or 96 hours. The SSOE was mixed with an equal amount of media (v/v) and 0.5 mL of this (media and SSOE) was added (up to the scleral rim), leaving the top of the cornea exposed to air; this mixture was changed every 24 hours. At the end of each study time point, the corneas were processed as described herein.

Histopathological evaluation of corneal sections, and measurement of epithelial degradation and epithelial healing was performed. Fixed corneal tissues were embedded in paraffin, 5 μm sections were sliced and stained with hematoxylin and eosin (H&E). The corneal sections were evaluated microscopically for epithelial degradation as described herein. The epithelial degradation was measured in the complete length of the cornea and the percentage epithelial degradation was calculated (length of the cornea with epithelial degradation/total length of the cornea×100). Epithelial healing (thickness of the epithelial layer after degradation at 96 hours post-CP exposure) was measured by selecting at least five randomly field throughout length of cornea with five measurements for each field at 40× magnification as reported earlier.

Apoptotic cell death detection was detected via TUNEL staining. Detection and quantification of apoptotic cell death in the corneal epithelial and stromal layers was carried out using the DeadEnd Colorimetric terminal deoxynucleotidyl transferase (tdt)-mediated dUTP-biotin nick end labeling (TUNEL) system, according to manufacturer protocol. Quantification of TUNEL positive cells was assessed in ten randomly selected fields (40× magnification), and an apoptotic cell index was calculated.

Immunoblot analysis included corneal protein lysates being prepared from the frozen corneal sections and subjected to SDS-PAGE, transferred to nitrocellulose membranes, blocked, and probed with appropriate primary antibodies for p-γH2AX, followed by incubation with peroxidase conjugated appropriate secondary antibody as published. Equal protein loading in each well was confirmed by stripping and re-probing the membranes with β-actin. After scanning, the bands were subjected to densitometric analysis employing the ImageJ Program (NIH, Bethesda, Md.).

Cytokine array was used to quantify inflammatory markers. Quantification of the protein levels of different inflammatory markers was carried out using the Quantibody® Rabbit Cytokine Array 1, as per manufacturer protocol. Briefly, the cytokine array slides were air dried for 1-2 hours and were blocked using the sample diluent for 30 min. Further, 100 μl of corneal protein lysate (75 μg) or standard cytokines were added in the dilution buffer in each well, and incubated overnight with the array slide at 4° C. Next, 80 μl of the detection antibody cocktail (biotinylated) was added, after washing with wash buffers for five times, and incubated for 1-2 hours at room temperature. After washing, 80 μl of Cy3 equivalent dye-conjugated streptavidin was added and incubated in the dark room for one hour. After the washing step, slides were sent for scanning, and analysis by RayBiotech (Norcross, Ga.).

Data were analyzed using one-way analysis of variance (one-way ANOVA) to get the statistically significant difference in control versus CP and SSOE treated groups, with Tukey t-test for multiple comparisons (GraphPad Prism 8 software). Difference was considered significant if the P value was <0.05. Data are presented as the mean±standard error of mean (SEM).

Embodiments described herein showed that the application of SSOE (55% w/v PFD) to the HCE cells increased cell viability and wound healing (scratch assay), and reversed CP induced DNA damage, apoptotic cell death, and oxidative stress markers. Results involving the ex-vivo rabbit cornea exposed the cornea to CP (700 nmol) for two hours and cultured in media with or without SSOE (55% w/v PFD) for 24 hours and 96 hours, and further evaluated the effect of SSOE treatment on CP-induced histopathological and molecular markers. To determine the frequency of SSOE treatment to the cornea, an oxygen sensor was used to measure the oxygen in the cornea of the ex vivo rabbit eyes. The readings revealed that oxygen within the anterior chamber increased from a baseline of ˜17 Torr to ˜120 Torr after the SSOE treatment. Oxygen within the anterior chamber dropped to ˜60 Torr after ˜7.5 hours and returned to baseline after ˜20 hours (FIG. 1C). Hence, the SSOE treatment was carried out every 24 hours in the corneal organ culture.

Further, SSOE treatment embodiments enhanced the healing of corneal epithelial layer at 96 hours post CP exposure. The rabbit corneas were exposed to CP (700 nmol) for two hours and cultured in the media with or without SSOE for 24 hours or 96 hours, and further evaluated for the effect of SSOE treatment on CP-induced corneal epithelial layer degradation. Results showed that CP treatment resulted in 86% degradation of the epithelial layer; however, SSOE treatment did not abrogate the CP-induced epithelial layer (85% degradation was observed in CP+SSOE group) at 24 hours post CP exposure. At 96 hours post exposure, some cellular proliferation of the degraded epithelial layer was observed indicating a healing effect of SSOE treatment on the corneal epithelial layer. Quantification of the epithelial layer thickness showed that CP exposure resulted in a significant decrease in the epithelial thickness (12.47 μm) as compared to control (27.45 μm) due to degradation. SSOE treatment resulted in increased proliferation of the layer resulting in an epithelial thickness of 18.62 μm. However, this effect was not significant as compared to the CP exposed epithelial layer at 96 hours post CP exposure.

Results also showed SSOE treatment reversed CP-induced increase in epithelial cell and keratocytes death at 96 h post CP exposure. Apoptosis is one of the early cellular responses after alkylating vesicating agents' exposure. In vitro studies in HCE cells described herein showed that CP exposure induced apoptotic cell death, and the SSOE treatment increased cell viability. Therefore, the epithelial apoptotic cell death was evaluated using TUNEL assay following CP exposure to the ex vivo rabbit cornea, and further assessed the effect of SSOE treatment on the CP-induced apoptotic epithelial cell death. The representative pictures of TUNEL-stained cornea sections showed that CP exposure induced a significant increase in apoptotic cell death in the corneal epithelial layer at both 24 hours and 96 hours post exposure. Quantification of TUNEL stained corneal sections revealed that CP exposure resulted in 30% and 39% TUNEL-positive epithelial cells compared to only 2% and 1% in the control groups after 24 hours and 96 hours post CP exposure, respectively. The SSOE treatment did not have an effect on the CP-induced epithelial cell death at 24 hours post-exposure. However, SSOE treatment resulted in a significant (93%) reversal in CP-induced epithelial apoptotic cell death at 96 hours post exposure.

The apoptotic death of keratocytes in the stoma following CP exposure to the ex vivo rabbit cornea was evaluated as was the effect of SSOE treatment on CP-induced apoptosis of keratocytes. Similar to the corneal epithelial cells, the representative TUNEL-stained corneal stromal sections showed that CP exposure induced a significant increase in keratocyte apoptosis at both 24 hours and 96 hours post exposure. Quantification of keratocytes demonstrated that CP exposure resulted in 33% and 53% keratocyte death compared to 1% and 3% in control groups at 24 hours and 96 hours post CP exposure, respectively. The SSOE treatment did not show any mitigation of CP-induced keratocyte apoptosis at 24 hours post exposure. However, SSOE treatment resulted in a 37% reversal in CP-induced keratocyte apoptosis at 96 hours post exposure.

Results further showed SSOE treatment reversed CP-induced DNA-damage, and CP-induced increases in cytokines and inflammatory markers related to cytokine signaling. As described herein, CP exposure in HCE cells caused cell death and DNA damage evidenced by the increase in the phosphorylation of H2A.X (Ser139), a marker for double stranded DNA breaks. Therefore, the phosphorylation levels of H2A.X (Ser139) were evaluated following exposure of the ex vivo rabbit cornea to CP. CP exposure caused a 1.7- and 7.3-fold increase in the phosphorylation of H2A.X compared to control at 24 hours and 96 hours post exposure, respectively. SSOE treatment reversed the CP-induced increase in the phosphorylation levels of H2A.X at both the time points. However, a stronger reversal (38%) was observed at 96 hours post-CP exposure as compared to reversal (12%) at 24 hours post exposure.

Cellular degradation and cell death by injury due to chemical exposures like vesicating chemical agents results in an inflammatory response. The levels of inflammatory markers in CP exposed corneal tissues were assessed using cytokine antibody array. At 24 hours and 96 hours post CP exposure, changes in inflammatory cytokines and other markers related to cytokine signaling were observed. At 24 hours post CP exposure, increased levels of interleukin (IL) 1α (2.61-fold), IL1β (4.16-fold), IL8 (1.26-fold), IL17A (7.46-fold), IL21 (1.7-fold), Leptin (3.21-fold), Macrophage inflammatory protein (MIP-1β; 5.44-fold), Matrix metallopeptidase 9 (MMP9; 2.61-fold), Neural Cell Adhesion Molecule 1 (NCAM1; 4.91-fold), and Tumor necrosis factor-α (TNFα; 4.61-fold), were observed as compared to their controls. Similarly, at 96 hours post CP exposure, increased levels of IL1α (1.8-fold), IL1β (1.4-fold), IL17A (2.0-fold), IL21 (1.4-fold), Leptin (1.3-fold), MIP-1β (1.4-fold), MMP9 (1.4-fold), NCAM1 (1.9-fold), TNFα (1.62-fold) were observed as compared to their controls.

Further, the effect of SSOE treatment on the reversal of CP-induced changes in the cytokines and inflammatory markers related to cytokine signaling was evaluated. At 24 hours post CP exposure, SSOE treatment reversed the CP-induced increases in IL17A (28%), IL21 (34%), Leptin (22%), MIP-1β (16%), MMP9 (11%), NCAM1 (47%), and TNFα (11%). At 96 hours post CP exposure, SSOE treatment reversed the CP-induced increases in IL1β (17%), IL17A (25%), Leptin (74%), MIP-1β (98%), MMP9 (86%), and TNFα (46%).

In addition to oxidative stress, hypoxia from chemical exposure is one of the major causes of delayed wound healing that leads to inflammation and tissue damage. Wound healing requires high metabolic activity that results in a high oxygen demand. Increased oxygen induces wound healing by stimulating several processes including phagocytosis, degradation of necrotic wound tissue, collagen production, decrease in inflammation, and by regulating macrophage infiltration. Oxygen ameliorates pathophysiological consequences to promote wound healing.

Results of embodiments described herein indicate that PFD-based SSOE, a topical oxygen application, is an effective therapeutic modality to supplement the corneal oxygen supply to promote re-epithelialization and enhance wound healing leading to the reversal of CP-induced corneal injury. Embodiments demonstrated that CP exposure to the cornea induced epithelial layer degradation, epithelial cell and keratocytes apoptotic death, caused DNA damage, and increased the expression of cytokines and inflammatory markers related to cytokine pathway. These corneal injury markers were used to further test the therapeutic potential of SSOE.

SSOE treatment of embodiments enhanced the epithelial wound healing and reversed the CP-induced epithelial cell and keratocytes apoptotic cell death at 96 hours post CP exposure. SSOE treatment also strongly mitigated the CP-induced DNA damage at 96 hours post CP exposure, and CP-induced increases in the expression of cytokines at both 24 hours and 96 hours post CP exposure. The ex vivo rabbit corneal organ culture described herein is a useful and cost-effective model to study corneal wound healing, and in the screening and identification of therapies against vesicant-induced ocular injuries. The vesicant corneal injury in the ex vivo rabbit corneal cultures were found to parallel those observed in the in vivo rabbit cornea. Hence, the ex vivo rabbit corneal injury model with CP provides a valuable tool to study the pathophysiology and mechanistic aspects of the CP corneal injury as well as be successfully employed to screen effective therapies.

Embodiments described herein in HCE cells showed that SSOE application every 24 hours is safe as it did not cause cell death and DNA damage, but instead promoted cell viability. The present embodiments indicated SSOE treatment enhanced corneal wound healing and mitigated CP-induced corneal apoptosis, DNA damage and inflammatory response. These outcomes support results of embodiments in HCE cells described herein indicating the therapeutic potential of SSOE in reversing CP-induced DNA damage, cell death and oxidative stress marker Embodiments also showed that SSOE treatment abrogates the CP-induced MMP9 expression, suggesting that MMP9 could be playing a critical role in CP-induced corneal injury, and is a possible target for SSOE. Oxygen therapy mitigates the inflammatory cytokines' expression in various pathophysiological conditions to enhance angiogenesis and wound repair The SSOE treatment in the ex vivo rabbit cornea also mitigated CP-induced inflammatory cytokines, indicating the possible mechanism involved in SSOE mediated wound healing in CP induced corneal injury.

It should be observed that the embodiments described in detail herein in accordance with the present invention reside primarily in combinations of method steps and chemical components related to a supersaturated oxygen emulsion (SSOE) comprising PFD. The above description of embodiments of the SSOE and corresponding treatment methods, test methods and results is not intended to be exhaustive or to limit the embodiments described to the precise form disclosed. While specific embodiments of, and examples for, the SSOE and corresponding treatment methods are described herein for illustrative purposes, various equivalent modifications are possible within the scope of the teachings described herein. The elements and acts of the various embodiments described above can be combined to provide further embodiments. These and other changes can be made to the SSOE and corresponding treatment methods in light of the above detailed description.

The numerical values cited in the specific embodiment are illustrative rather than limiting. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

In general, in the following claims, the terms used should not be construed to limit the SSOE and corresponding treatment methods to the specific embodiments disclosed in the specification and the claims but should be construed to include all embodiments and methods that operate under the claims. Accordingly, the SSOE and corresponding treatment methods are not limited by the disclosure, but instead the scope of the SSOE and corresponding treatment methods is to be determined entirely by the claims.

While certain aspects of the SSOE and corresponding treatment methods are presented below in certain claim forms, the inventors contemplate the various aspects of the SSOE and corresponding treatment methods in any number of claim forms. Accordingly, the inventors reserve the right to add additional claims after filing the application to pursue such additional claim forms for other aspects of the SSOE and corresponding treatment methods.

What is claimed is:

1. A method comprising:
configuring an oxygenated material for application to an alkali burn in an ocular region, wherein the oxygenated material includes a supersaturated oxygen emulsion (SSOE) including perfluorodecalin (PFD) homogenized with a phospholipid and an emulsifying wax, wherein the SSOE comprises a viscosity configured to deliver the SSOE to a site of the alkali burn and sustain a partial pressure of oxygen at the site at a level a plurality of times greater than an ambient partial pressure of oxygen for a plurality of hours; and
configuring a pressurized dispensing canister to house and dispense the SSOE directly on the site.

2. The method of claim 1, wherein the ocular region includes at least one of corneal tissue, limbal tissue, ocular adnexal tissue, retinal tissue, ocular nerve tissue, and vitreous compartments.

3. The method of claim 1, comprising configuring the SSOE to at least one of facilitate healing of the alkali burn and preserve ocular tissue.

4. The method of claim 1, comprising configuring the SSOE to include a percentage weight of PFD of approximately 25 percent of a total volume (w/v).

5. The method of claim 4, wherein the phospholipid includes Phospholipon 90H and the emulsifying wax includes Polawax.

6. The method of claim 4, comprising supersaturating the SSOE with medical grade oxygen gas.

7. The method of claim 6, wherein a diameter of the emulsified nanoparticles of the SSOE is approximately in a range of 50 nm to 300 nm.

8. The method of claim 1, comprising configuring the SSOE to be sustained on the site at a level approximately four times the partial pressure of oxygen in the atmosphere for more than two hours.

9. The method of claim 1, comprising configuring the SSOE with a pH of approximately 6.7.

10. The method of claim 1, wherein the SSOE promotes corneal epithelial wound healing.

11. The method of claim 1, wherein the SSOE at least one of mitigates and prevents ocular hypoxia.

12. The method of claim 1, wherein the SSOE at least one of mitigates and prevents cell apoptosis.

13. The method of claim 1, wherein the SSOE at least one of mitigates and prevents tissue inflammation.

14. The method of claim 1, wherein the SSOE at least one of mitigates and prevents at least one of reduced optical transparency and cataract formation.

15. The method of claim 1, wherein the SSOE at least one of mitigates and prevents anterior chamber exudation in the anterior chamber between cornea and lens.

16. The method of claim 1, wherein the SSOE at least one of mitigates and prevents tissue fibrosis and scar formation.

17. A compound comprising an oxygenated material configured for application to an alkali burn in an ocular region, wherein the oxygenated material includes a supersaturated oxygen emulsion (SSOE) including perfluorodecalin (PFD) homogenized with a phospholipid and an emulsifying wax, wherein the SSOE comprises a viscosity configured to deliver the SSOE to a site of the alkali burn and sustain a partial pressure of oxygen at the site at a level a plurality of times greater than an ambient partial pressure of oxygen for a plurality of hours.
